(19) United States
(12) Patent Application Publication
(10) Pub. No.: US 2025/0108160 A1
(43) Pub. Date: Apr. 3, 2025

(54) SYSTEMS AND METHODS FOR MANAGING MESSAGES SENT TO USER OF AN INSULIN PUMP SYSTEM

(71) Applicant: Tandem Diabetes Care, Inc., San Diego, CA (US)

(72) Inventors: Andrew KAPLAN, San Diego, CA (US); Frank ATORE, San Diego, CA (US)

(73) Assignee: Tandem Diabetes Care, Inc., San Diego, CA (US)

(21) Appl. No.: 18/477,384

(22) Filed: Sep. 28, 2023

(51) Int. Cl. A61M 5/142

(52) U.S. Cl. CPC A61M 5/142; A61M 2005/14208; A61M 2205/502

(57) ABSTRACT

Systems and methods are provided for managing communication with a user of an insulin pump system for improving insulin pump operation and/or a user experience relating to the insulin delivery system. The insulin pump system may analyze data from a user of a certain insulin pump and/or data from users of different insulin pumps and determine patterns relating to such use and indicative of certain user experiences. Based on the detected patterns and/or events associated with the patterns or user experiences, user messages may be proactively sent a user device and/or smart device of the user. The user messages may be designed to improve a user experience and/or prevent a negative outcome. For example, a message may be a training video, may include a reminder to adjust pump operation for a certain activity, and/or may include an alert to reorder more supplies.

### TECHNICAL FIELD

This technology generally relates to the field of insulin pump systems. For example, systems and methods are provided herein for diabetes management using an insulin pump system.

### BACKGROUND

Nearly 1 in 10 individuals in the United States are affected by diabetes. As technology advances, techniques for glucose monitoring and even insulin delivery so too develop and evolve. To manage the condition, historically many of these individuals were required to administer a blood draw, for example a needle prick in the fingertip to analyze the blood to determine blood glucose levels. If the blood glucose level did not satisfy a threshold, the individual may have to administer an insulin shot, meaning an injection of insulin into the subcutaneous tissue using a needle and syringe.

With advances in monitoring technology, continuous glucose monitoring using a wearable patch including a small needle now provides on demand glucose monitoring for adjusting pump operation based on the glucose measurements. Additionally, pump operation may be adjusted based on information about the user's activities including food consumption, sleep information and any other relevant information. Sensors and/or smart devices may also collect information that may be relevant to and/or useful for managing blood glucose levels. Such information may be used to inform pump operation and/or to improve the user experience generally for those affected by diabetes.

While users of insulin pumps may generate a significant amount of data and data may be generated about such users from smart devices and from the insulin pump itself, it is difficult to process and/or analyze this large volume of varied data to make informed decisions about operation of the insulin pump and/or otherwise improve the user experience. Further, a user may only have access to their own data and trends and/or patterns detected in data and thus would not have access to other data (e.g., anonymized data) that could improve the user experience and/or pump operation for such user.

Accordingly, there is a need for improved systems and methods for managing user communications relating to operation of an insulin pump to improve pump operation, blood glucose levels, and/or the user experience.

### SUMMARY

Provided herein are systems and methods for managing communication with a user of an insulin pump system for improving insulin pump operation and/or user experience for the insulin delivery system. The system and methods may include determining data relevant to insulin delivery and/or diabetes management for multiple users and analyzing such data to determine user experiences such as negative experiences. The system may collect user data to detect indictors of such user experiences (e.g., events associated with the user experiences) and may send a user message relating to such user experience to the user (e.g., to be presented on the user's mobile device, insulin pump, and/or smart device). The user message may be training or an alert designed to proactively adjust operation of the pump or user behavior to avoid the negative user experience. Alternatively, an alert for training or user support may be generated when such indicators are detected.

A method is provided herein for managing alerts communicated to a user of an insulin delivery pump. The method may include determining a pattern indicative of a user experience based on a plurality of user data relating to use of plurality of insulin delivery pumps different from the insulin delivery pump, determining an event corresponding to the pattern, determining a user record corresponding to the user of the insulin delivery pump, determining the user record includes the event, determining a user message associated with the event, the user message corresponding to the user experience, causing, based on determining the user record includes the event, a user device associated with the insulin delivery pump to present the user message, and populating, automatically, the user record with a record of the user message.

The method may further include determining the user message based on the user experience and associating the user message with the event in a database and/or datastore. The user message may be associated with the event and may include using the database to determine if the user message is associated with the event. The database and/or datastore may include a plurality of events associated with a plurality of user messages. The user message may include textual, video, haptic, and/or audio information. The user message may be a training video adapted to be displayed on the user device. The user message may be a reminder to change the insulin pump infusion site. The method may further include determining sensor data. The event may be determined based on the sensor data. The event may be determined based on an amount of elapsed time and/or a set number of event indicators (e.g., 3 alerts that cartridge door is open). The method may further include determining a second user message associated with the user record, and determining, before causing the user device to present the user message, that the user message does not conflict with the second user message.

A system is provided herein for managing alerts communicated to a user of an insulin delivery pump and/or a healthcare provider or other agent of the user. The system may include memory configured to store computer-executable instructions, and at least one computer processor configured to access memory and execute the computer-executable instructions to determine a pattern indicative of a user experience based on a plurality of user data relating to use of plurality of insulin delivery pumps different from the insulin delivery pump, determine an event corresponding to the pattern, determine a user record corresponding to the user of the insulin delivery pump, determine the user record includes the event, determine a user message associated with the event, the user message corresponding to the user experience, cause, based on determining the user record includes the event, a user device associated with the insulin delivery pump to present the user message, and populate, automatically, the user record with a record of the user message.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1 illustrates an exemplary insulin pump system for analyzing user data of a user of a wearable insulin pump and data from other individuals to determine, detecting trends, and generating user messages.

FIG. 2 illustrates an exemplary process flow for determining patterns in data from insulin pump users and sending a user message when an event related to a pattern is detected in user data.

FIG. 3 illustrates an exemplary data flow including user data and aggregated user data processed by a remote device to inform a message sent to a user device and smart device.

FIG. 4 illustrates an exemplary data flow depicting user data such as error data that is recorded in a user record by remote device and used to inform a message sent to a user device.

FIG. 5 illustrates a schematic block diagram of a remote device in accordance with one or more exemplary embodiments of the disclosure.

The foregoing and other features of the present invention will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

The present disclosure is directed to systems and methods for managing communication with a user of an insulin pump system for improving insulin pump operation and/or user experience for the insulin delivery system. For example, data relevant to insulin delivery using an insulin pump and/or diabetes management for multiple users may be analyzed to determine user experiences such as negative experiences. Trends and/or patterns may be identified relating to the user experiences and/or events may be identified for detecting such patterns and/or trends.

Information generated by a user, an insulin pump of a user, or other sensors and/or smart devices of a user may be analyzed to determine such events or other indictors of a user experience identified in the aggregate data. Upon determining the presence of such event or other indicator, a user message may be sent to the user (e.g., to be presented on the user's mobile device, insulin pump, and/or smart device). The user message may be training or an alert designed to proactively adjust operation of the pump or user behavior to avoid the negative user experience.

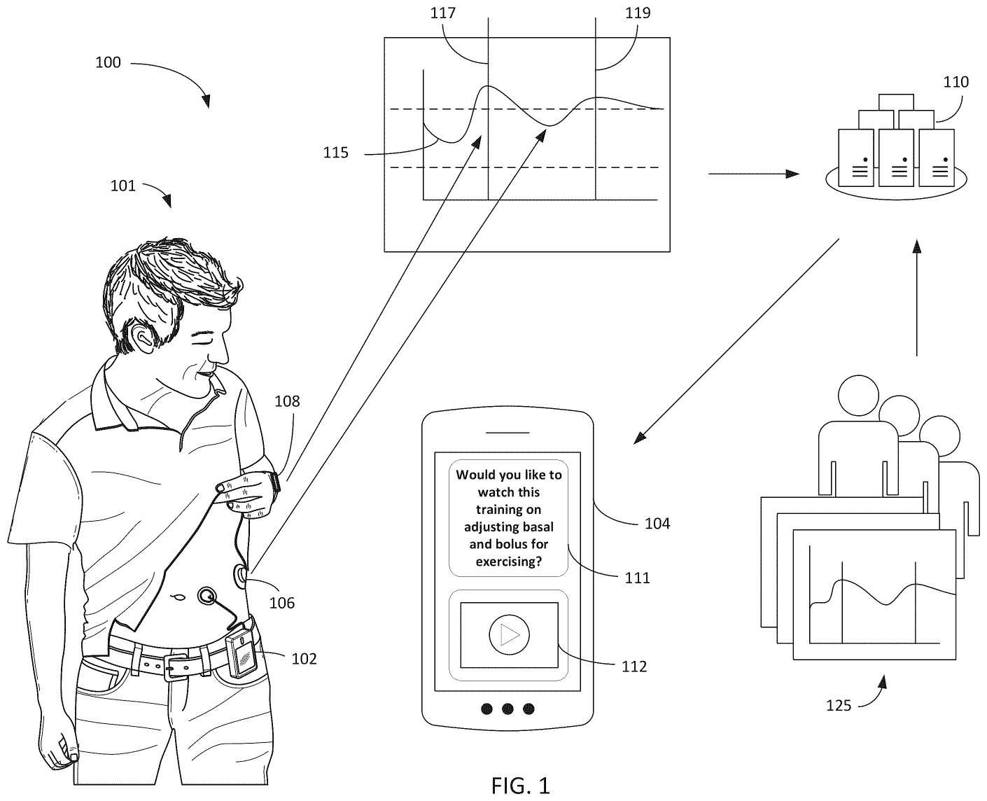

Referring now to FIG. 1, an insulin pump system for managing communications with the user to improve the user experience and/or operation of the insulin pump is depicted. Insulin pump system 100 may include insulin pump 102 and server 110. Insulin pump system 100 may also include or communicate with glucose monitor 106, user device 104 (e.g., smart phone) and/or smart device 108. Insulin pump system 100 may include greater or fewer devices than those illustrated in FIG. 1.

Insulin pump 102 may, in one example, be the same as or similar to a t:slim X2™ insulin pump available from Tandem Diabetes Care, Inc. of San Diego, California and/or the insulin pump described in U.S. Patent App. Pub. No. 2014/0276423, published on Sep. 18, 2014 and assigned to Tandem Diabetes Care, Inc., and/or the insulin pump described in U.S. Patent App. Pub. No. 2011/014461, published on Jun. 16, 2011 and assigned to Tandem Diabetes Care, Inc., each of which are hereby incorporated by reference in their entirety. However, it is understood that insulin pump 102 may be any type of insulin pump for delivering insulin to the user and having the functionality described herein.

Insulin pump 102 may include a pump housing which may include a display and may house a pump designed to pump insulin into a user. For example, insulin pump 102 may be connected to a delivery component which may include tubing and/or a delivery patch having a needle or cannula for delivering insulin into a subcutaneous area of the user at an infusion site. Insulin pump 102 may include a compartment for holding a volume of insulin and may selectively deliver an amount of insulin (e.g., a bolus) to the user via the pump and delivery component 114.

Insulin pump 102 may include one or more processors and memory as well as a communication chip for communicating with one or more devices via a suitable wireless connection (e.g., Bluetooth, Bluetooth Low Energy (BLE), Wi-Fi Direct, any other near field communication protocol, and/or the like). For example, insulin pump 102 may communicate wirelessly with user device 104, smart device 108, glucose monitoring sensor 106 and/or other devices. Insulin pump 102 may optionally communicate directly with remote device 110, glucose monitoring sensor 106 and/or any other devices or communicate with such devices via user device 104 and/or smart device 108.

User device 104 and/or smart device 108 may be any suitable smart device, smart phone, mobile phone, tablet, laptop, wearable, charging device, and/or smart sensor. The user device and/or smart device may include a computer processor, memory, microphone, display (e.g., touchscreen) and/or a communication unit which may facilitate communication with any other devices (e.g., healthcare provider device, smart sensors such as glucose monitoring sensor 106, and/or any other devices). User device 104 and/or smart device 108 may communicate with any devices in insulin pump system 100 and any other devices via any suitable wired or wireless system (e.g., Wi-Fi, cellular network, Bluetooth, Bluetooth Low Energy (BLE), near field communication protocol, etc.). Additionally, or alternatively, messages and/or alerts may be sent to a computing device of a healthcare provider or other agent of the user.

Remote device 110 may be any computing device having a processor, memory and a communication unit. For example, remote device 110 may be one or more servers, datastores, or the like. Remote device 108 may communicate with any devices in insulin pump system 100 and any other devices via any well-known wired or wireless system (e.g., Wi-Fi, cellular network, Bluetooth, Bluetooth Low Energy (BLE), near field communication protocol, etc.).

Insulin pump 102 may generate data relating to the operation of insulin pump 102, such as bolus information, dosing information, insulin delivery information, battery information, insulin supply information, errors or alerts relating to pump operation, pump settings information, and the like. Insulin pump 102 may share this information with remote device 110. For example insulin pump 102 may send this information to user device 104 and user device may send the information to remote device 110. In one example, the remote device may be an electronic medical records (EMR) device and/or server that maintains EMR.

Smart device 108 may similarly generate data relating to the user, which may be informative of the user's general health and/or physiological response to insulin delivery. For example, smart device 108 may generate heartrate data, exercise data (e.g., step count), sleep data, temperature data, electrocardiogram (ECG data), and/or any other data that may be sensed or determined. In one example, smart device 108 may be a smart watch or wearable device.

Insulin pump 102, user device, 104, remote device 110, and/or smart device 108 may be in communication with glucose monitoring device 106, which may be any suitable continuous glucose monitoring sensor such as the Dexcom G7 Continuous Glucose Monitoring (CGM) system available from Dexcom, Inc. of San Diego, California and/or any other blood glucose monitoring device. For example, glucose monitoring device 106 may share glucose information (e.g., blood glucose measurements at specific times) with remote device 110, either directly with remote device 110 or indirectly with remote device 110 via user device 104.

As shown in FIG. 1, glucose data 115 may be sent from glucose monitoring device 106 to remote device 110 (e.g., via user device 104). Further exercise data 117 and 119 may be sent from smart device 108 to remote device 110. Glucose data 115 may include measurements for the blood glucose levels of the user (e.g., user 101) over time. Exercise information 117 and 119 may be heart rate information, for example, and may indicate times during which exercise occurred (e.g., times for which a high heart rate was detected).

Remote device 110 may process glucose data 115 and exercise data 117 and 119 of user 101 and may determine exercise data 117 and 119 coincides temporally with highs in blood glucose levels. Remote device 110 may also process aggregated data 125, which may be anonymized blood glucose and exercise data from multiple different users. In one example, such aggregated data may all come from the same health care provider, from users with similar medical history, demographics, and/or having any other similarity with user 101.

Remote device 110 may detect a similar pattern in aggregated data 125. Remote device 110 may further determine that when users adjust their bolus and/or basal for exercising, the blood glucose measurements during and/or after exercising are within a normal range for a greater percentage of time. As a result, when remote device detects the pattern in glucose data 115 and/or exercise data 117 and 119, remote device 110 may automatically determine user messaging for adjusting user behavior and/or use of insulin pump 102 (e.g., insulin pump settings) to result in improved blood glucose levels.

In the example illustrated in FIG. 1, remote device 110 may determine that additional training on adjusting bolus and/or basal or otherwise adjusting pump settings during exercising has historically resulted improved blood glucose levels for other users (e.g., users corresponding to aggregated data 125). As result, upon detecting the pattern of high blood glucose levels during exercise, remote device 110 may send a message to user device 104, which may be associated with insulin pump 102 (e.g., on a user account associated with user 101).

User device 104 may include text message 111 including text asking the user if the user would like to watch a training on adjusting basal and bolus for exercising. This may be a text message for example or may appear in a message in a user application associated with insulin pump 102. The message presented on user device 104 may optionally include video 112, which may be a video training for adjusting basal and/or bolus for exercising. Alternatively, a link to a video on a mobile application or website may be shared on user device 104. In yet another example, recommended pump settings may be presented with a request to adjust pump operation based on the recommended pump settings.

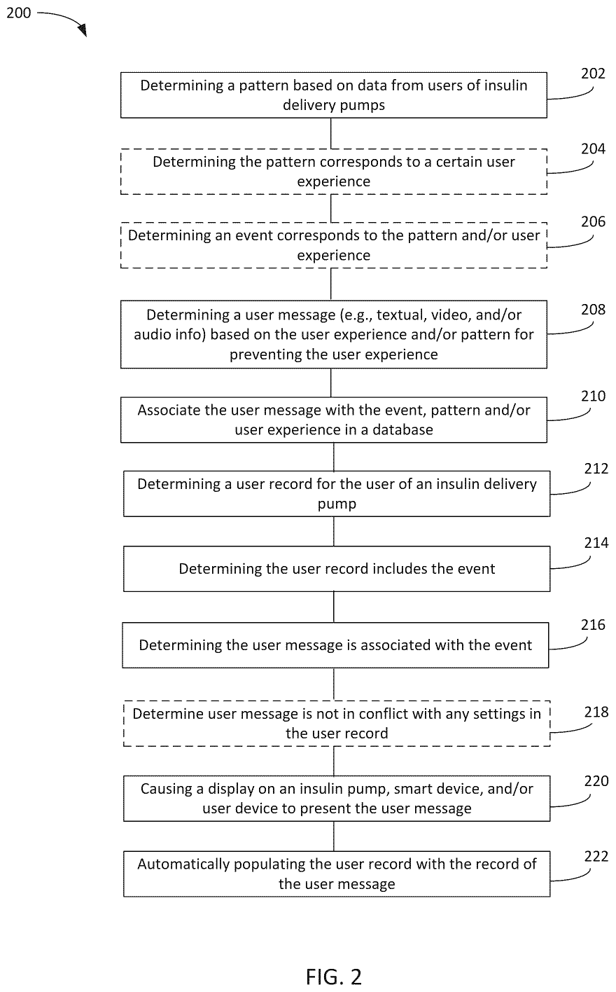

Referring now to FIG. 2, an exemplary process flow for determining patterns in data from insulin pump users and sending a user message when an event related to a pattern or event is detected in user data is illustrated. Process flow may be performed by a remote device (e.g., remote device 110 of FIG. 1). Alternatively, some or all of the blocks of process flow 200 in FIG. 2 may be performed in a distributed manner across any number of devices (e.g., remote device, mobile device, smart device, etc.). Some or all of the operations of process flow 200 may be optional and may be performed in a different order.

To initiate process flow 200, at block 202, computer-executable instructions stored on a memory of a device, such as a remote device, may be executed to determine a pattern based on data (aggregated data) for multiple users of insulin delivery pumps. For example, data may include blood glucose measurements over time, exercise data (e.g., heart rate, step count, ECG information, duration, etc.), sleep data (e.g., REM cycles, duration), food data (e.g., nutritional information such as carbohydrates calories, sugar, fat etc.), pump operation data (e.g., infusion site information, bolus information, basal information, alerts, battery information, etc.).

Exemplary patterns include or relate to high or low glucose levels during or after an activity (e.g., exercising, eating, sleeping), decreasing blood glucose time in normal range with increasing days at the same infusion site, alerts corresponding to connecting pump with other devices (e.g., continuous glucose monitors), pump installation, insulin supply levels, battery operation, and the like. It is understood that any other pattern may be detected by remote device 110 and may be proactively addressed with a user message to improve the user experience and/or pump operation. In one example, insulin supply levels and/or historical data including supply orders (e.g., purchases) may be analyzed to determine purchase and/or use patterns. Based on such patterns, the system may determine intervals for which orders are typically made and may generate an alert and/or reminder to make a purchase and/or may otherwise initiate an order or send a message to approve a purchase order based on such interval (e.g., every 3 weeks). The order may be based on an amount of supplies typically ordered. In another example, similar alerts, reminders and/or messages may be generated for purchasing and/or replacing a key components of the pump.

At optional block 204, computer-executable instructions stored on a memory of a device, such as a remote device, may be executed to determine the pattern corresponds to a certain user experience. For example, the user experience may be prolonged out-of-range blood glucose levels, infusion site issues, pump setting issues, exercise settings issues, sleep settings issues, and/or general pump operation issues. The user experience may be a negative user experience that may be desirable to avoid. It is understood that any other user experiences may be determined.

At optional block 206, computer-executable instructions stored on a memory of a device, such as a remote device, may be executed to determine that an event corresponds to pattern detected and/or the user experience detected. For example, the event may be the occurrence of the pattern, the occurrence of the pattern a certain number of times, a sensor measurement, a detection of an anomaly and/or abnormality, an elapsed amount of time, or any other event, activity, and/or data.

At block 208, computer-executable instructions stored on a memory of a device, such as a remote device, may be executed to determine a user message based on the user experience and/or pattern. For example, the user message may be designed to cause a user to adjust user behavior or insulin pump operation to prevent and otherwise alleviate the user experience. The message may be, for example, textual information, a video or image, and/or an audio file).

At block 210, computer-executable instructions stored on a memory of a device, such as a remote device, may be executed to determine associate the user message with the event, pattern, and/or user experience. For example, a database and/or datastore may maintain the associations between the event, pattern, and/or user experience. The term database is used herein to mean a database, a datastore, and/or the like. Steps 202 to 210 may be repeated for each pattern detected based on data from users of insulin delivery pumps. At optional block 212, computer-executable instructions stored on a memory of a device, such as a remote device, may be executed to determine a user record (e.g., user data) associated with a certain insulin (e.g., insulin pump identifier). The user record may include information about the user such as name, weight, height, medical information, physiological information, biological information, insulin delivery information (e.g., settings), blood glucose information, sensor information, and the like.

At block 214, computer-executable instructions stored on a memory of a device, such as a remote device, may be executed to determine that the user record includes an event. For example, the user record indicates that the user did a certain activity at a certain time or for a certain duration, that a period of time has elapsed, that a sensor measurement exceeds a threshold, or the like. Alternatively, the remote device may determine that an event occurred without consulting the user record (e.g., remote device may automatically be informed by the insulin pump, smart device, and/or glucose monitoring device).

At block 216, computer-executable instructions stored on a memory of a device, such as a remote device, may be executed to determine the user message is associated with the event. For example, the event may be exercise and the message may be a reminder to adjust bolus or basal or training relating adjusting bolus and/or basal. For example, a database and/or datastore may be consulted to determine that the event, pattern, and/or user experience determined in block 214 is associated with a certain user message. At block 218, computer-executable instructions stored on a memory of a device, such as a remote device, may be executed to determine that the user message associated with the event is not in conflict. For example, the user record may include a setting preventing messages at a certain time or a setting a limiting the number of messages for a given time period, for example.

At block 220, computer-executable instructions stored on a memory of a device, such as a remote device, may be executed to cause the display on the insulin pump, smart device, and/or user device to present the user message. It is understood that the user message on one device may be different than the user message sent to another device. For example, one device may present a text message and another device may present a video. At block 222, computer-executable instructions stored on a memory of a device, such as a remote device, may be executed to automatically populate the user record with a record of the user message.

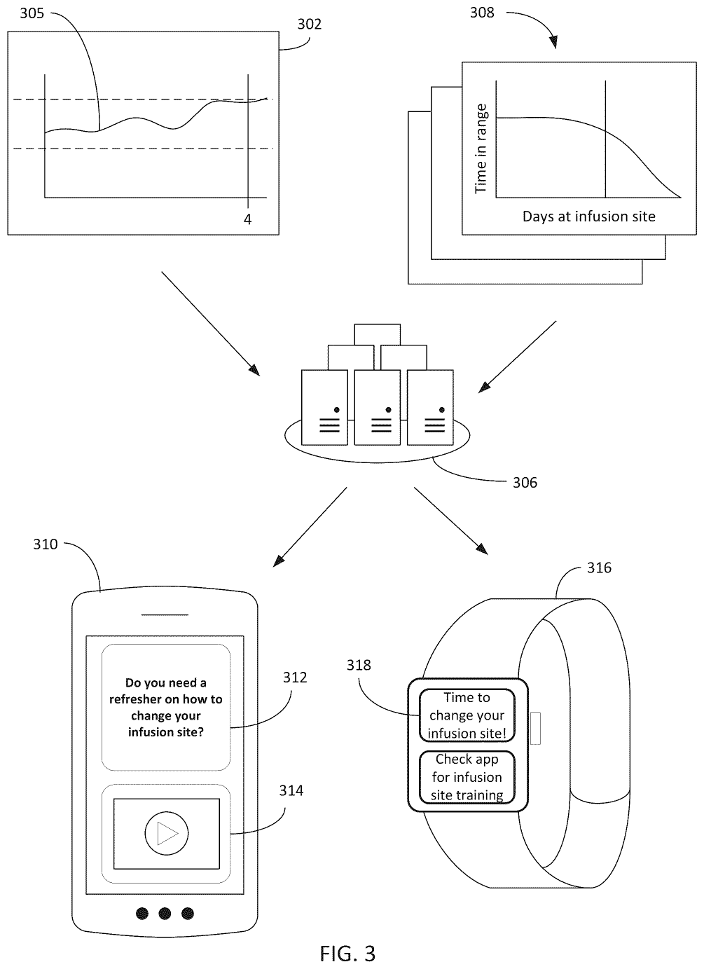

Referring now to FIG. 3, an exemplary data flow depicting user data and aggregated user data used to inform a message sent to a user device and smart device is illustrated. More specifically, FIG. 3 illustrates an exemplary use case in which remote device 306 determines based on aggregated data 308 to alert the user that it is time to change the infusion site of an insulin pump and sends a training video to the user on how to change the infusion site.

As shown in FIG. 3, user data 302 may be generated by a blood glucose monitor and/or a smart sensor or smart device and may be sent to remote device 306 (e.g., via a user device such as user device 310 and/or smart device 316). Remote device 306, user device 310, and/or smart device 316 may be the same as or similar to remote device 110, user device 104 and/or smart device 108 of FIG. 1, respectively.

User data 302 may include blood glucose data 305 generated by a glucose monitoring device. Such blood glucose data 305 may include measurements of the user's blood glucose levels over time. User data 302 may show several days' worth of blood glucose measurements and may indicate that as time goes on, the blood glucose levels increase. Aggregate user data 308 may also be sent to or otherwise accessed by remote device 310. Aggregate user data 308 may indicate that for multiple different users that the amount or percent of time user's blood glucose levels are within a normal expected range (e.g., not high and not low as defined by certain threshold amounts) decreases over time for the days that the insulin pump remains at the same infusion site and decreases more rapidly after a certain day (e.g., the fifth day at the same infusion site).

Based on the pattern identified by remote device 306, remote device may determine an event associated with the negative experience of decreased blood glucose levels in range. For example, the remote device may determine after day 4 the time in range of the blood glucose levels begin to drop rapidly. As a result, the event may be the elapsed time of 4 days at the same infusion site.

Remote device 305 may process user data 302 and may determine from user data 302 that the infusion site has been the same for 4 days. This information may otherwise be determined from the user record. Based on this determination, remote device 306 may consult a database and/or datastore associating the event of elapsed time on the 4^{th} day with one or more messages.

As shown in FIG. 3, messages may be sent to user device 310 and messages may be also sent to smart device 316. For example, remote device 306 may cause user device 310 to send message 312 asking the user if they need a refresher on how to change their infusion site and may also cause user device to present training message 314 which may be a video training for changing the infusion site. Similarly, remote device 306 may send message 318 to smart device 316, which may alert the user that it is time to change the infusion site and further suggest that the user check the user application for the infusion site training. It is understood that any messages relevant to the user experience detected by remote device 306 may be sent to user device 310 and/or smart device 316.

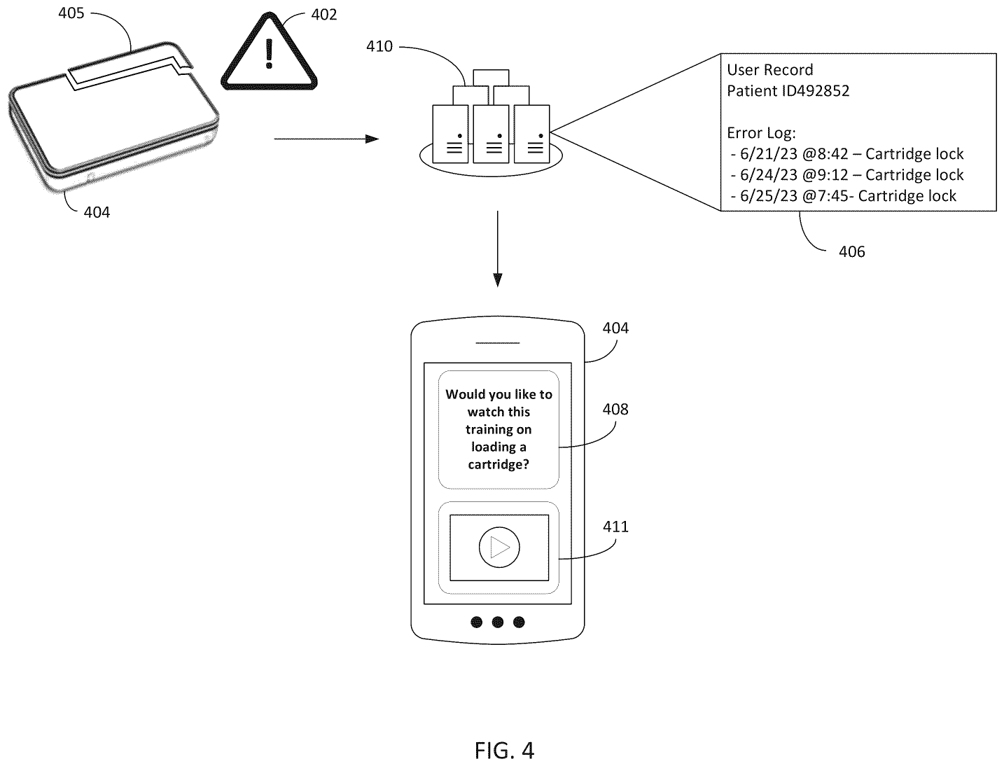

Referring now to FIG. 4, an exemplary data flow depicting user data such as error data used to inform a message sent to the user device. For example, remote device 410, which may be the same as remote device 110 of FIG. 1, may receive error alert 402 which may indicate that a cartridge on insulin device 404 was improperly loaded or was left open for an extended period of time. Insulin device 404 may be the same as insulin device 102 of FIG. 1. Cartridge 405 may be removed from time-to-time to refill insulin pump 405 with insulin and may click into or otherwise lock into insulin pump 404.

Remote device 410 may maintain user record 406 which may keep a log of all alerts and/or errors messages or signals received from insulin device 404. User record 406 may indicate three error alerts regarding cartridge 405 were received in a short period of time. Based on these error alerts, remote device 410 may determine that the user of insulin pump 404 is having a difficult time installing insulin and/or loading the cartridge.

Remote device 410 may determine a pattern of errors and upon the a number of error messages, may determine a user message to improve the user experience. For example, after three error messages within a week, remote device 410, which may be the same as remote device 110, may send user device 404 messages 408 and 411. Message 408 may include text asking the user if the user would like to watch a training on loading a cartridge. Message 411 may be a training video for loading a cartridge.

It is understood that a similar process may be used for different use cases. For example, an elapsed time or low insulin levels may be used as an event to trigger a reminder alert to reorder supplies (e.g., insulin). In another example, records of connection issues with a glucose monitoring device may trigger a message with a training video for syncing the insulin pump with a glucose monitoring device. It is understood that any other message may be sent to improve a user experience and/or pump operation based on a detected pattern and/or occurrence of an event.

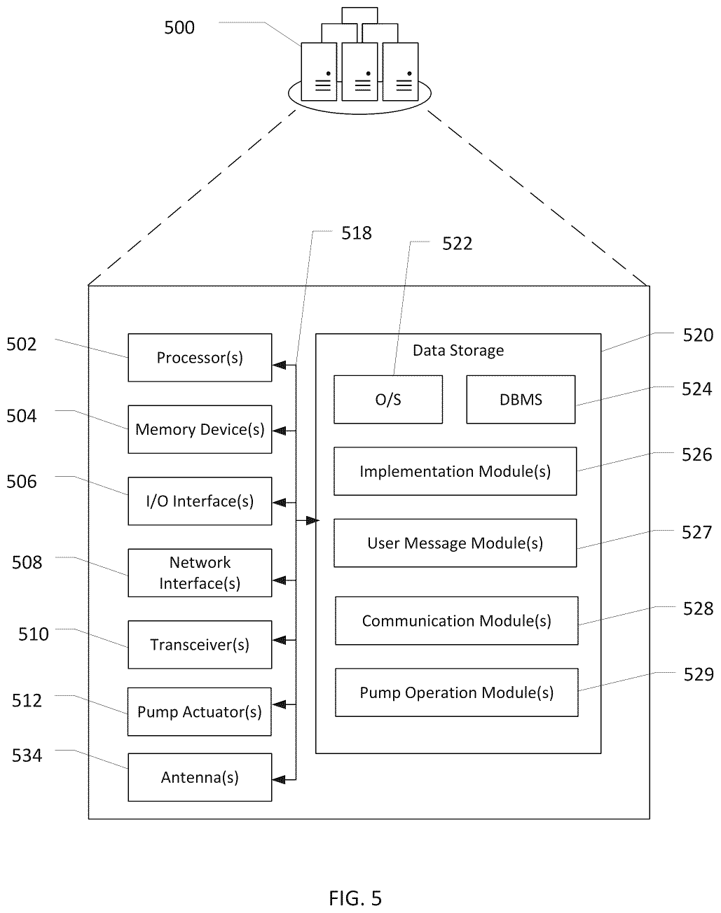

FIG. 5 is a schematic block diagram of illustrative remote device 500, which may be in communication with one or more periphery (e.g., user device, smart device, etc.) and/or remote devices, is illustrated. Remote device 500 may be the same or similar to remote device 110 of FIG. 1 or otherwise one or more of the remote devices of FIGS. 2-4. It is understood that remote device 500 may alone or together with one or periphery or remote devices perform one or more of the operations of remote device 500. For example, a user device (e.g., user device 104 of FIG. 1) may perform one or more of the operations and tasks of the remote device.

Remote device 500 may be designed to communicate with one or more periphery devices, remote devices, smart devices, computing devices, servers, other systems, or the like. Remote device 500 may be designed to communicate via one or more networks. Such network(s) may include, but are not limited to, any one or more different types of communications networks such as, for example, near field communication networks, cable networks, public networks (e.g., the Internet), private networks (e.g., frame-relay networks), wireless networks, cellular networks, telephone networks (e.g., a public switched telephone network), or any other suitable private or public packet-switched or circuit-switched networks.

In an illustrative configuration, remote device 500 may include one or more processors 502, one or more memory devices 506 (also referred to herein as memory 506), one or more input/output (I/O) interface(s) 506, one or more network interface(s) 508, one or more transceiver(s) 510, one or more pump actuator(s) 512, one or more antenna(s) 534, and data storage 520. Remote device 500 may further include one or more bus(es) 518 that functionally couple various components of the remote device 500.

The bus(es) 518 may include at least one of a system bus, a memory bus, an address bus, or a message bus, and may permit exchange of information (e.g., data (including computer-executable code), signaling, etc.) between various components of the remote device 500. The bus(es) 518 may include, without limitation, a memory bus or a memory controller, a peripheral bus, an accelerated graphics port, and so forth. The bus(es) 518 may be associated with any suitable bus architecture including.

The memory 506 may include volatile memory (memory that maintains its state when supplied with power) such as random access memory (RAM) and/or non-volatile memory (memory that maintains its state even when not supplied with power) such as read-only memory (ROM), flash memory, ferroelectric RAM (FRAM), and so forth. Persistent data storage, as that term is used herein, may include non-volatile memory. In various implementations, the memory 506 may include multiple different types of memory such as various types of static random access memory (SRAM), various types of dynamic random access memory (DRAM), various types of unalterable ROM, and/or writeable variants of ROM such as electrically erasable programmable read-only memory (EEPROM), flash memory, and so forth.

The data storage 520 may include removable storage and/or non-removable storage including, but not limited to, magnetic storage, optical disk storage, and/or tape storage. The data storage 520 may provide non-volatile storage of computer-executable instructions and other data. The memory 506 and the data storage 520, removable and/or non-removable, are examples of computer-readable storage media (CRSM) as that term is used herein. The data storage 520 may store computer-executable code, instructions, or the like that may be loadable into the memory 506 and executable by the processor(s) 502 to cause the processor(s) 502 to perform or initiate various operations. The data storage 520 may additionally store data that may be copied to memory 506 for use by the processor(s) 502 during the execution of the computer-executable instructions. Moreover, output data generated as a result of execution of the computer-executable instructions by the processor(s) 502 may be stored initially in memory 506, and may ultimately be copied to data storage 520 for non-volatile storage.

The data storage 520 may store one or more operating systems (O/S) 522; one or more optional database management systems (DBMS) 524; and one or more program module(s), applications, engines, computer-executable code, scripts, or the like such as, for example, one or more implementation modules 526, one or more user message modules 527, one or more communication modules 528, and/or one or more pump operation modules 529. Some or all of these modules may be sub-modules. Any of the components depicted as being stored in data storage 520 may include any combination of software, firmware, and/or hardware. The software and/or firmware may include computer-executable code, instructions, or the like that may be loaded into the memory 506 for execution by one or more of the processor(s) 502. Any of the components depicted as being stored in data storage 520 may support functionality described in reference to correspondingly named components earlier in this disclosure.

Referring now to other illustrative components depicted as being stored in data storage 520, O/S 522 may be loaded from data storage 520 into memory 506 and may provide an interface between other application software executing on remote device 500 and hardware resources of the remote device 500. More specifically, O/S 522 may include a set of computer-executable instructions for managing hardware resources of remote device 500 and for providing common services to other application programs (e.g., managing memory allocation among various application programs). In certain example embodiments, O/S 522 may control execution of the other program module(s) for content rendering. O/S 522 may include any operating system now known or which may be developed in the future including, but not limited to, any server operating system, any mainframe operating system, or any other proprietary or non-proprietary operating system.

Optional DBMS 524 may be loaded into the memory 506 and may support functionality for accessing, retrieving, storing, and/or manipulating data stored in memory 506 and/or data stored in data storage 520. DBMS 524 may use any of a variety of database models (e.g., relational model, object model, etc.) and may support any of a variety of query languages. DBMS 524 may access data represented in one or more data schemas and stored in any suitable data repository including, but not limited to, databases (e.g., relational, object-oriented, etc.), file systems, flat files, distributed datastores in which data is stored on more than one node of a computer network, peer-to-peer network datastores, or the like.

I/O interface(s) 506 may facilitate the receipt of input information by remote device 500 from one or more I/O devices as well as the output of information from remote device 500 to the one or more I/O devices. The I/O devices may include any of a variety of components such as a touchscreen display; an audio output device for producing sound, such as a speaker; an audio capture device, such as a microphone; buttons and/or dials; and so forth. Any of these components may be integrated into remote device 500 or may be separate.

Remote device 500 may further include one or more network interface(s) 508 via which remote device 500 may communicate with any of a variety of other systems, platforms, networks, devices, and so forth. Network interface(s) 508 may enable communication, for example, with one or more wireless routers, one or more host servers, one or more web servers, and the like via one or more of networks.

Antenna(s) 534 may include any suitable type of antenna depending, for example, on the communications protocols used to transmit or receive signals via antenna(s) 534. Non-limiting examples of suitable antennas may include directional antennas, non-directional antennas, dipole antennas, folded dipole antennas, patch antennas, multiple-input multiple-output (MIMO) antennas, or the like. Antenna(s) 534 may be communicatively coupled to one or more transceivers 510 or radio components to which or from which signals may be transmitted or received. Antenna(s) 534 may include, without limitation, a cellular antenna for transmitting or receiving signals to/from a cellular network infrastructure, an antenna for transmitting or receiving Wi-Fi signals to/from an access point (AP), a Global Navigation Satellite System (GNSS) antenna for receiving GNSS signals from a GNSS satellite, a Bluetooth antenna for transmitting or receiving Bluetooth signals including BLE signals, a Near Field Communication (NFC) antenna for transmitting or receiving NFC signals, a 900 MHz antenna, and so forth.

Transceiver(s) 510 may include any suitable radio component(s) for, in cooperation with the antenna(s) 534, transmitting or receiving radio frequency (RF) signals in the bandwidth and/or channels corresponding to the communications protocols utilized by remote device 500 to communicate with other devices. Transceiver(s) 510 may include hardware, software, and/or firmware for modulating, transmitting, or receiving—potentially in cooperation with any of antenna(s) 534—communications signals according to any of the communications protocols discussed above including, but not limited to, one or more Wi-Fi and/or Wi-Fi direct protocols, as standardized by the IEEE 802.11 standards, one or more non-Wi-Fi protocols, or one or more cellular communications protocols or standards. Transceiver(s) 510 may further include hardware, firmware, or software for receiving GNSS signals. Transceiver(s) 510 may include any known receiver and baseband suitable for communicating via the communications protocols utilized by the remote device 500. The transceiver(s) 510 may further include a low noise amplifier (LNA), additional signal amplifiers, an analog-to-digital (A/D) converter, one or more buffers, a digital baseband, or the like.

Referring now to functionality supported by the various program module(s) depicted in FIG. 6, implementation module(s) 526 may include computer-executable instructions, code, or the like that responsive to execution by one or more of the processor(s) 502 may perform functions including, but not limited to, overseeing coordination and interaction between one or more modules and computer executable instructions in data storage 520, determining user actions, determining actions associated with user interactions, determining actions associated with user input, initiating commands locally or at periphery and/or remote devices, and the like.

The user message module(s) 527 may include computer-executable instructions, code, or the like that responsive to execution by one or more of the processor(s) 502 may perform functions including, but not limited to analyzing user data and and/or aggregated data to determine patterns, determining events related to the patterns, determining user experiences relating to the patterns, and/or determining user messages relating to the patterns, events, and/or user experiences and causing user devices and/or smart devices to present the messages.

Communication module(s) 528 may include computer-executable instructions, code, or the like that responsive to execution by one or more of the processor(s) 502 may perform functions including, but not limited to, communicating with one or more devices, for example, via wired or wireless communication, communicating with user devices (e.g., mobile devices), smart devices, remote devices, charging devices, computing devices, smart sensors and/or any other devices.

Pump operation module(s) 529 may include computer-executable instructions, code, or the like that responsive to execution by one or more of the processor(s) 502 may perform functions including, but not limited to, overseeing, monitoring, and/or operating the pump and pump actuators for select delivery of a volume of insulin based on pump parameters for the insulin delivery pump (e.g., pump settings).

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Certain aspects of the disclosure are described above with reference to block and flow diagrams of systems, methods, apparatuses, and/or computer program products according to example embodiments. It will be understood that one or more blocks of the block diagrams and flow diagrams, and combinations of blocks in the block diagrams and the flow diagrams, respectively, may be implemented by execution of computer-executable program instructions. Likewise, some blocks of the block diagrams and flow diagrams may not necessarily need to be performed in the order presented, or may not necessarily need to be performed at all, according to some embodiments. Further, additional components and/or operations beyond those depicted in blocks of the block and/or flow diagrams may be present in certain embodiments.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

Program module(s), applications, or the like disclosed herein may include one or more software components, including, for example, software objects, methods, data structures, or the like. Each such software component may include computer-executable instructions that, responsive to execution, cause at least a portion of the functionality described herein (e.g., one or more operations of the illustrative methods described herein) to be performed.

A software component may be coded in any of a variety of programming languages. An illustrative programming language may be a lower-level programming language such as an assembly language associated with a particular hardware architecture and/or operating system platform. A software component including assembly language instructions may require conversion into executable machine code by an assembler prior to execution by the hardware architecture and/or platform.

Another example programming language may be a higher-level programming language that may be portable across multiple architectures. A software component including higher-level programming language instructions may require conversion to an intermediate representation by an interpreter or a compiler prior to execution.

Other examples of programming languages include, but are not limited to, a macro language, a shell or command language, a job control language, a script language, a database query or search language, or a report writing language. In one or more example embodiments, a software component including instructions in one of the foregoing examples of programming languages may be executed directly by an operating system or other software component without having to be first transformed into another form.

A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may include other custom-developed application software, operating system functionality (e.g., device drivers, data storage (e.g., file management) routines, other common routines, and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages, but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in the flow diagrams to be performed. These computer program instructions may also be stored in a CRSM that upon execution may direct a computer or other programmable data processing apparatus to function in a particular manner, such that the instructions stored in the computer-readable storage medium produce an article of manufacture including instruction means that implement one or more functions or operations specified in the flow diagrams. The computer program instructions may also be loaded onto a computer or other programmable data processing apparatus to cause a series of operational elements or steps to be performed on the computer or other programmable apparatus to produce a computer-implemented process.

Additional types of CRSM that may be present in any of the devices described herein may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the information and which can be accessed. Combinations of any of the above are also included within the scope of CRSM. Alternatively, computer-readable communication media (CRCM) may include computer-readable instructions, program module(s), or other data transmitted within a data signal, such as a carrier wave, or other transmission. However, as used herein, CRSM does not include CRCM.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

It should be understood that any of the computer operations described herein above may be implemented at least in part as computer-readable instructions stored on a computer-readable memory. It will of course be understood that the embodiments described herein are illustrative, and components may be arranged, substituted, combined, and designed in a wide variety of different configurations, all of which are contemplated and fall within the scope of this disclosure.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A method for managing alerts communicated to a user of an insulin delivery pump, the method comprising:
determining a pattern indicative of a user experience based on a plurality of user data relating to use of plurality of insulin delivery pumps different from the insulin delivery pump;
determining an event corresponding to the pattern;
determining a user record corresponding to the user of the insulin delivery pump;
determining the user record comprises the event;
determining a user message associated with the event, the user message corresponding to the user experience;
causing, based on determining the user record comprises the event, a user device associated with the insulin delivery pump to present the user message; and
populating, automatically, the user record with a record of the user message.

2. The method of claim 1, further comprising:
determining the user message based on the user experience; and
associating the user message with the event in a database.

3. The method of claim 2, wherein determining the user message is associated with the event comprises using the database to determine the user message is associated with the event.

4. The method of claim 2, wherein the database comprises a plurality of events associated with a plurality of user messages.

5. The method of claim 1, wherein the user message comprises textual, video, haptic, and/or audio information.

6. The method of claim 5, wherein the user message is a training video adapted to be displayed on the user device.

7. The method of claim 1, wherein the user message is a reminder to change the insulin pump infusion site.

8. The method of claim 1, further comprising determining sensor data, wherein the event is determined based on the sensor data.

9. The method of claim 1, wherein the event is determined based on an amount of elapsed time and/or a set amount of event indicators.

10. The method of claim 1, further comprising:
determining a second user message associated with the user record; and
determining, before causing the user device to present the user message, that the user message does not conflict with the second user message.

11. A system for managing alerts communicated to a user of an insulin delivery pump, the system comprising:
memory configured to store computer-executable instructions; and
at least one computer processor configured to access memory and execute the computer-executable instructions to:
determine a pattern indicative of a user experience based on a plurality of user data relating to use of plurality of insulin delivery pumps different from the insulin delivery pump;
determine an event corresponding to the pattern;
determine a user record corresponding to the user of the insulin delivery pump;
determine the user record comprises the event;
determine a user message associated with the event, the user message corresponding to the user experience;
cause, based on determining the user record comprises the event, a user device associated with the insulin delivery pump to present the user message; and
populate, automatically, the user record with a record of the user message.

12. The system of claim 11, wherein the at least one computer processor is further configured to access memory and execute the computer executable instructions to:
determine the user message based on the user experience; and
associate the user message with the event in a database.

13. The system of claim 12, wherein determining the user message is associated with the event comprises using the database to determine the user message is associated with the event.

14. The system of claim 12, wherein the database comprises a plurality of events associated with a plurality of user messages.

15. The system of claim 11, wherein the user message comprises textual, video, haptic, and/or audio information.

16. The system of claim 11, wherein the user message is a training video adapted to be displayed on the user device.

17. The system of claim 11, wherein the user message is a reminder to change the insulin pump infusion site.

18. The system of claim 11, wherein at least one computer processor is further configured to access memory and execute the computer executable instructions to determine sensor data, and wherein the event is determined based on the sensor data.

19. The system of claim 11, wherein the event is determined based on an amount of elapsed time and/or a set amount of event indicators.

20. The system of claim 11, wherein the at least one computer processor is further configured to access memory and execute the computer executable instructions to:
determine a second user message associated with the user record; and
determine, before causing the user device to present the user message, that the user message does not conflict with the second user message.
